(19) United States
(12) Patent Application Publication
(10) Pub. No.: US 2021/0213435 A1
(43) Pub. Date: Jul. 15, 2021

(54) HOLLOW ZEOLITES CATALYSTS FOR THE PRODUCTION OF ALKL AROMATIC COMPOUNDS FROM AROMATIC HYDOCARBONS AND OLEFINS

(71) Applicant: SABIC Global Technologies B.V., Bergen op Zoom (NL)

(72) Inventors: Ugo Ravon, Thuwal (SA); Gregory Biausque, Thuwal (SA); Khalid Albahily, Thuwal (SA)

(21) Appl. No.: 15/999,766

(22) PCT Filed: Feb. 7, 2017
(86) PCT No.: PCT/IB2017/050656

§ 371 Date: Aug. 20, 2018

Related U.S. Application Data
(60) Provisional application No. 62/297,482, filed on Feb. 19, 2016.
(60) Provisional application No. 62/378,478, filed on Aug. 23, 2016.

(51) Int. Cl. B01J 29/80; B01J 29/035; C07C 2/66

(52) U.S. Cl. CPC B01J 29/80; B82Y 30/00; C07C 2/66; B01J 29/0356

(57) ABSTRACT

Supported catalysts, methods of making and using are described herein. A supported catalyst can include a metal nanostructure, an oxide, or an alloy thereof, having a Lewis acid active site capable of catalyzing the formation of an alkyl aromatic compound from an aromatic hydrocarbon and an olefin, and an inert hollow zeolite support. The inert hollow zeolite support has a peripheral shell with an exterior surface and an interior surface that defines and encloses a hollow space within the interior of the shell, where the metal nanostructure, or an oxide or an alloy thereof is comprised in the hollow space.

### CROSS REFERENCE TO RELATED APPLICATIONS

This application claims the benefit of priority of U.S. Provisional Patent Application No. 62/297,482 filed Feb. 19, 2016, and U.S. Provisional Patent Application No. 62/378,478 filed Aug. 23, 2016. The entire contents of each of the above-referenced disclosures are specifically incorporated herein by reference without disclaimer.

### BACKGROUND OF THE INVENTION

### A. Field of the Invention

The invention generally concerns a catalyst for the production of alkyl aromatic compounds from aromatic hydrocarbons and olefins. In particular, the invention concerns a catalyst that includes a metal nanostructure or an oxide or an alloy thereof having a Lewis acid active site capable of catalyzing alkyl aromatic formation from aromatic hydrocarbons and olefins, and a hollow zeolite support having a peripheral shell with an exterior surface and an interior surface that defines and encloses a hollow space within the interior of the shell, wherein the metal nanostructure, or an oxide or an alloy thereof, is comprised in the hollow space.

### B. Description of Related Art

Ethylbenzene (C₈H₁₀) is a raw material used in the production of chemical products, in particular, styrene, which in turn is used in the production of styrene polymers and copolymers. There are many processes to produce ethylbenzene with the alkylation reaction of ethylene (C₂H₄) and benzene (C₆H₆) in a catalytic environment being most widely used. Historically, catalysts employed in commercial applications included aluminum chloride (AlCl₃) or BF₃ as acidic catalysts. In more recent commercial applications zeolite-based acidic catalysts have been used (for example, synthetic zeolite MCM-22, which is a MWW type zeolite, or H-ZSM-5). U.S. Pat. No. 6,268,305 to Butler et al., describes the use of a solid, non-hollow, silicate catalyst having bimodal acidity designed to have weak acid sites and strong acid sites to catalyst the production of ethylbenzene from ethylene and benzene.

Many these types of conventional zeolite catalysts used in alkylation reactions include metal promoters and/or catalytic material. However, these catalysts suffer from deactivation, stability, and leaching of the catalytic material. By way of example, the catalytic material can be smaller than the pores of the zeolite allowing the catalytic material to diffuse through the pore, which diminishes the stability of the catalyst. Other problems associated with deactivation of zeolites containing catalytic material include poor dispersion of the catalytic material on the surface of the zeolite. Further, these acidic zeolite catalysts have sufficient amounts of strong acid sites to effect formation of byproducts (e.g., diethylbenzene, triethylbenzene, diphenyl ethane, or other polyalkylated benzenes). While many by-products can be separated from reaction product, proper disposal of these by-products adds to the cost and reduces the yield of the desired product ethylbenzene.

### SUMMARY OF THE INVENTION

A solution to the problems associated with the costs, deactivation, and degradation of alkylation catalysts has been discovered. The solution lies in the production of alternatives to the aforementioned acidic zeolite catalysts. In particular, it was surprisingly found that a metal nanostructure, an oxide or an alloy thereof, with a Lewis acid active site encapsulated in an inert or substantially inert zeolite support was capable of catalyzing the alkylation reaction of an aromatic hydrocarbon and olefin to form an alkyl aromatic compound, preferably an alkylation reaction to form ethylbenzene from benzene and ethylene. Alternatively, the alkylation reaction can be used to form isopropylbenzene (cumene) from benzene and propylene. Notably, the inert hollow zeolite support (i.e., a support that is substantially or completely non-reactive with the reactants and/or reactive intermediates, such as a support that is substantially or completely devoid of active acidic sites) has a peripheral shell with an exterior surface and an interior surface that defines and encloses a hollow space within the interior of the shell, and the metal nanostructure, or an oxide or an alloy thereof is comprised in the hollow space. Without wishing to be bound by theory, it is believed that the metal nanostructure or oxide or alloy thereof encapsulated in the inert hollow zeolite structure offers increased catalytic stability and efficiency in producing ethylbenzene or cumene. The size of the metal nanostructure, oxide or alloy thereof is believed to be sufficiently small to prevent coking yet sufficiently large enough to be retained inside the hollow zeolite structure, which can inhibit leaching of the metal nanostructure from the catalyst. The metal nanostructure, oxide, or alloy thereof, preferably does not include materials having Lewis basic sites (e.g., Column 1 metals, notably, potassium, sodium or magnesium). The inert zeolite can be any zeolite having a Si/Al ratio of 500 to infinity (∞), with infinity being pure silica zeolite. The inert zeolite can have a minimal amount or no acidic functionality. Furthermore, the inner and outer surfaces of the peripheral shell can have the same zeolitic framework, thereby having the same or substantially the same physical and chemical properties.

In a particular aspect of the present invention, a supported catalyst capable of catalyzing the alkylation of an aromatic hydrocarbon with an olefin to produce alkyl aromatic compounds is described. In a particular embodiment, the alkylation of benzene with ethylene to produce ethylbenzene is described. In another particular embodiment, the alkylation of benzene with propylene to produce cumene is described. The supported catalyst can include a metal nanostructure, an oxide, or an alloy thereof, having a Lewis acid active site capable of catalyzing the alkylation reaction of the aromatic hydrocarbon with the olefin (e.g., ethylbenzene formation from benzene and ethylene, cumene formation from benzene and propylene, etc.). The supported catalyst can have an inert or substantially inert hollow zeolite support having a peripheral shell with an exterior surface and an interior surface that defines and encloses a hollow space within the interior of the shell. The hollow zeolite support can be a single particle. Catalysts of the present invention can be a single particle or can include a plurality of such particles. The hollow zeolite support can be a *BEA, MFI, silicalite-1, or any combination thereof. In some embodiments, the *BEA zeolite includes at least one fluorine atom. A hollow zeolite particle can have a particle size of 20 nm to 300 nm, preferably 20 nm to 100 nm, more preferably 30 nm to 80 nm, or most preferably 50 nm to 60 nm. A catalyst having a plurality of zeolite particles can have a bimodal distribution of particles. A first distribution of particles can have an average particle size from 20 nm to 100 nm, preferably 30 nm to 80 nm, or more preferably 50 nm to 60 nm and a second distribution of particles can have an average particle size of greater than 100 nm to 300 nm. The thickness of the peripheral shell can be 5 nm to 30 nm, preferably 10 nm to 20 nm. The volume of the hollow space can be 5% to 90% of the initial particle volume (i.e., before the hollow space is formed). The average particle size of the metal nanostructure or oxide or alloy thereof can be 0.6 to 50 nm, preferably 0.6 to 30 nm, more preferably 0.6 to 15 nm, or most preferably ≤10. In preferred aspects, the metal nanostructure is larger than the zeolite pore size. Thickness and particle size can be measured using TEM. The single metal nanostructure, an oxide or an alloy thereof or a plurality of metal nanostructures, oxides or alloys thereof can be contained in the hollow space. The metal nanostructure, an oxide or an alloy thereof can be deposited on the interior surface of the peripheral shell and/or the size of the hollow space and the metal nanostructure, the oxide or the alloy thereof, are both larger than the average pore size of the pores in the hollow zeolite support. In some embodiments, the size of the hollow space is 20 nm to 100 nm, preferably 30 nm to 80 nm, or more preferably 50 nm to 60 nm. The metal nanostructure, oxide or alloy thereof can be a transition metal (e.g., vanadium (IV) oxide, vanadium (V) oxide, iron (II) or (III) oxide (preferably iron (III) oxide), and niobium (III) oxide), a post transition metal (e.g., aluminum (III) oxide, gallium (III) oxide and titanium (IV) oxide), or both having an oxidation state value from +2 to +7, preferably from +2 to +5, and more preferably from +2 to +3. In some instances, the crystal structure of the metal nanostructure oxide phases or phases is a mono oxide, a composite oxide, or a mixed oxide (e.g., a spinel, perovskite, pyrochlore, and the like). The supported catalyst can include 0.5 to 20 wt. %, preferably 1 to 10 wt. % of the metal nanostructure, oxide or alloy thereof and from 80 to 99.5 wt. % of the hollow zeolite support, based on the total weight of the supported catalyst. In a particular embodiment, the metal nanostructure is not an iron-potassium (FeK) containing metal nanostructure and/or the hollow zeolite support is not a ZSM-5 support.

In another aspect of the invention, a method for producing alkyl aromatic hydrocarbons is described. The method can include contacting any one of the supported catalysts described above or throughout the specification with an aromatic hydrocarbon and an olefin in a reaction zone under reaction conditions sufficient to produce an alkyl aromatic compound. Reaction conditions can include a temperature of about 150° C. to about 400° C., a pressure of about 5 bar to 70 bar and/or a gas hourly space velocity (GHSV) ranging from about 1000 to about 100,000 h⁻¹. In a preferred aspect, the catalyst is contacted with benzene and ethylene to produce ethylbenzene having the selectivity as shown in FIG. 6. In some instances, the catalyst is an iron encapsulated silicalite-1 catalyst, wherein the iron particle is comprised in an intra-particle hollow space of the silicalite-1 shell. In other instances, the catalyst is contacted with benzene and propylene to produce cumene.

In yet another aspect of the invention, a method of making the supported catalyst of as described above or throughout the specification is described. The method can include (a) obtaining a zeolite support; (b) obtaining a first suspension by suspending the zeolite support in an aqueous solution having a metal nanostructure precursor material for a sufficient period of time to impregnate the support with the precursor material and drying the first suspension to obtain an impregnated support; (c) obtaining a second suspension by suspending the impregnated support from step (b) in an aqueous solution comprising a templating agent and thermally treating the suspension to obtain a templated support; and (d) calcining the templated support to obtain the supported catalyst of the present invention. The metal nanostructure precursor material can be a metal nitrate, a metal amine, a metal halogen, a metal coordination complex, a metal sulfate, a metal phosphate hydrate, or combination thereof. Drying the first suspension to obtain the impregnated support in step (b) can include subjecting the first suspension to a temperature of 30° C. to 100° C., preferably 30° C. to 60° C., for 2 to 24 hours, preferably 2 to 6 hours. Thermally treating the second suspension to obtain the templated support in step (c) can include subjecting the second suspension to a temperature of 100° C. to 250° C., preferably 150° C. to 200° C., for 12 to 96 hours, preferably 24 to 48 hours. The calcining step (d) can include subjecting the templated support to a temperature of 400° C. to 600° C., preferably 450° C. to 550° C., for 3 to 10 hours, preferably 4 to 8 hours.

Systems for producing alkyl aromatic hydrocarbons (e.g., ethylbenzene, cumene, etc.) are also described. A system can include (a) an inlet for a reactant feed; (b) a reaction zone (e.g., a continuous flow reactor selected from a fixed-bed reactor, a fluidized reactor, or a moving bed reactor) that is configured to be in fluid communication with the inlet, wherein the reaction zone includes the supported catalyst of the present invention; and (c) an outlet configured to be in fluid communication with the reaction zone and configured to remove a product stream from the reaction zone. The reactant feed can include ethylene and benzene or propylene.

The following includes definitions of various terms and phrases used throughout this specification.

The phrases “hollow space” and “intra-particle hollow space” each refer to a hollow space or void in within the interior surface of a zeolite shell. FIG. 1A provides a non-limiting example of a particle of the present invention that includes a single hollow space or intra-particle hollow space. FIG. 1B provides a non-limiting example of a particle of the present invention that includes two intra-particle hollow spaces.

The phrase “inter-particle space” refers to a space or void that is created when multiple particles are contacted with one another and spaces or voids are created between the outer surfaces of such particles. FIG. 1C provides a non-limiting example of a plurality of particles of the present invention, each having a single hollow space or intra-particle hollow space, that form inter-particle spaces or voids between the outer surfaces of such particles.

The term “catalyst” refers to a single hollow zeolite particle or a plurality of hollow zeolite particles positioned adjacent to each other in a catalytic bed and/or shaped into a form that can catalyze a chemical reaction. FIGS. 1A-1E provide non-limiting examples of catalysts of the present invention.

“Nanostructure” refers to an object or material in which at least one dimension of the object or material is equal to or less than 1000 nm (e.g., one dimension is 1 to 1000 nm in size). In a particular aspect, the nanostructure includes at least two dimensions that are equal to or less than 1000 nm (e.g., a first dimension is 1 to 1000 nm in size and a second dimension is 1 to 1000 nm in size). In another aspect, the nanostructure includes three dimensions that are equal to or less than 1000 nm (e.g., a first dimension is 1 to 1000 nm in size, a second dimension is 1 to 1000 nm in size, and a third dimension is 1 to 1000 nm in size). The shape of the nanostructure can be of a wire, a particle (e.g., having a substantially spherical shape), a rod, a tetrapod, a hyper-branched structure, a tube, a cube, or mixtures thereof. “Nanostructures” include particles having an average diameter size of 1 to 1000 nanometers. In a particular instance the nanostructure is a nanoparticle. The particle size of the nanostructure can be measured using known techniques. Non-limiting examples include transmission electron spectroscopy (TEM), scanning electron microscopy (SEM) preferably TEM.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The term “substantially” and its variations are defined to include ranges within 10%, within 5%, within 1%, or within 0.5%. By way of example, an inter hollow support of the present invention can have an overall surface area that includes less than 10%, less than 5%, less than 1%, less than 0.5% or no acidic sites that react with ethylene and benzene.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The catalysts of the present invention can “comprise,” “consist essentially of,” or “consist of” particular ingredients, components, compositions, etc. disclosed throughout the specification. With respect to the transitional phase “consisting essentially of,” in one non-limiting aspect, a basic and novel characteristic of the catalysts of the present invention are (1) the metal nanostructures, oxides, or alloys thereof having a Lewis acid active site that are encapsulated in an inert hollow zeolite structure and (2) their use in catalyzing ethylbenzene formation from ethylene and benzene.

The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present invention may become apparent to those skilled in the art with the benefit of the following detailed description and upon reference to the accompanying drawings.

FIG. 1A is an illustration of an embodiment of a cross-sectional view of an encapsulated nanostructure in a hollow zeolite with the nanostructure contacting the inner surface of the hollow space.

FIG. 1B is an illustration of an embodiment of a cross-sectional view of an encapsulated nanostructure in a hollow zeolite with the nanostructure not contacting the inner surface of the hollow space.

FIG. 1C is an illustration of an embodiment of a cross-sectional view of a plurality of encapsulated nanostructures in a hollow zeolite.

FIG. 1D is an illustration of an embodiment of a cross-sectional view of two encapsulated nanostructures in two intra-particle hollow spaces of a hollow zeolite.

FIG. 1E is an illustration of an embodiment of a cross-sectional view of a plurality of zeolite particles, each having an intra-particle hollow space, that form inter-particle spaces between the outer surfaces of the particles.

FIG. 2A is a schematic of a method to produce the catalyst of the present invention with a single metal nanostructure, oxide or alloy thereof.

FIG. 2B is a schematic of a method to produce the catalyst of the present invention with multiple metal nanostructures, oxides or alloys thereof.

FIG. 3 is a schematic of a system to produce alkyl aromatic hydrocarbons (e.g., ethylbenzene, cumene, etc.).

FIGS. 4A and 4B are Transmission Electron Microscopy (TEM) images of the catalyst of the present invention with scales of 200 nm (FIG. 4A) and 20 nm (FIG. 4B).

FIG. 5 is an Energy Dispersive X-ray spectroscopy (EDAX) pattern of the catalyst of the present invention.

FIG. 6 is a gas chromatogram of ethylbenzene produced using the catalyst of the present invention.

FIG. 7 is a gas chromatogram of ethylbenzene produced using a comparative H-ZSM-5 (Si/Al=30).

While the invention is susceptible to various modifications and alternative forms, specific embodiments thereof are shown by way of example in the drawings and may herein be described in detail. The drawings may not be to scale.

### DETAILED DESCRIPTION OF THE INVENTION

The currently available commercial catalysts used to produce alkyl aromatic hydrocarbons such as ethylbenzene from ethylene and benzene are prone to leaching, growth of carbon residuals (e.g., coke and carbon whiskers), and sintering. Further, the zeolite supports include active acidic sites that can catalyze the formation of by-products. These problems can lead to inefficient catalyst performance and ultimately failure of the catalyst after relatively short periods of use. This can lead to inefficient alkyl aromatic hydrocarbon (e.g., ethylbenzene, cumene, etc.) production as well as increased costs associated with such production.

An unexpected discovery has been made that avoids problems associated with deactivation of alkyl aromatic hydrocarbon catalysts and/or the formation of unwanted by-products. In particular, the catalyst of the present invention are based on encapsulating a transition metal or post transition metal, oxide or alloy thereof, having a Lewis acid active site in an inert zeolite support. Notably, the catalyst does not rely on the zeolite to catalyze the reaction and/or does not include any Lewis base components (e.g., a Column 1 metal, oxide or alloy thereof). The method of making the catalyst allows for creation of a hollow space in the zeolite and subsequent encapsulation of the metal nanostructure or a plurality of metal nanostructures, oxides or alloys thereof in the hollow zeolite. The method also allows control of the size the metal nanostructure. Without wishing to be bound by theory, it is believed that by focusing the alkylation reaction on the metal nanostructure rather than the inert zeolite support, less by-products will be produced, thereby increasing the efficiency of the catalyst in the production of alkyl aromatic hydrocarbons such as ethylbenzene and cumene. Further, it is believed that because the metal nanostructure size is larger than the pore size of the zeolite, the metal nanostructures cannot diffuse out of the zeolite so they remain inside the hollow space of the zeolite created. Thus, the metal nanostructure, oxide or alloy thereof cannot grow or sinter, and hence size is maintained (i.e., sintering is prevented) and/or leaching of the metal from zeolite is inhibited. Moreover, because the size of the metal nanostructure is reduced, the formation of coke can be inhibited. Furthermore, the methods used to prepare the catalysts of the present invention allow tuning of the size of the metal nanostructures as well. Further, the thickness of the hollow zeolite shell can also be tuned as desired.

These and other non-limiting aspects of the present invention are discussed in further detail in the following sections.

### A. Catalyst Structure

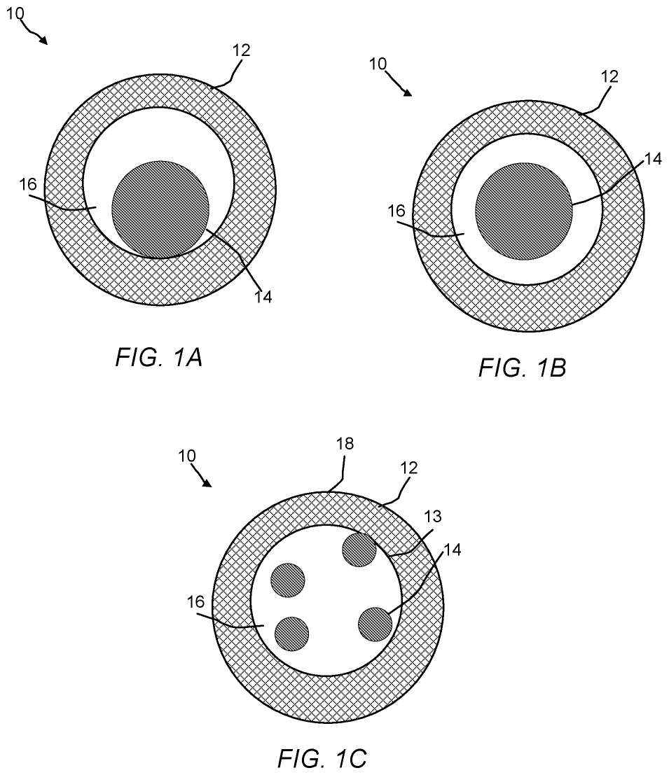

The metal nanostructure/hollow zeolite structure of the present invention includes a metal nanostructure, oxide or alloy thereof (“metal nanostructure”) contained within a hollow space that is present in the zeolite. FIGS. 1A through 1E are cross-sectional illustrations of catalyst material 10 having an encapsulated metal nanostructure/hollow zeolite structure. The catalyst material 10 has a zeolite shell 12, a metal nanostructure 14 and hollow space 16. In some embodiments, a portion of the nanostructure 14 (e.g., M¹, M² and/or M³) can be deposited on the surface of the zeolite (not shown). As discussed in detail below, the hollow space 16 can be formed by removal of a portion of the zeolite core during the making of the catalyst material. As shown in FIG. 1A, the metal nanostructure 14 contacts a portion of the inner wall of hollow space 16. As shown in FIG. 1B, the bimetallic or trimetallic nanostructure 14 does not contact the walls of the hollow space 16. As shown in FIG. 1C, multiple metal nanostructures 14 are in hollow space 16 with some metal nanostructures 14 touching the inner wall of the hollow space. FIG. 1D depicts the intra-particle hollow zeolite particle 10 having two intra-particle hollow spaces. In certain aspects, 1% to 99%, 10% to 80%, 20% to 70%, 30% to 60%, 40% to 50% or any range or value there between of the nanostructures fills the hollow space 16. The pore size of the catalyst is the same or similar to the pore size of the starting zeolite (e.g., about 5.5 Å). A volume space of the hollow space can be about 30 to 80%, 40 to 70%, or 50 to 60% of the zeolite particle volume or 30%, 35%, 40%, 45%, 50%, 55%, 60%, 65%, 70%, 75%, 80% or any value or range there between. The volume of the hollow space can be measured using transmission electron microscopy (TEM). The particle size of a single particle hollow zeolite support can be 20 to 300 nm, 30 to 80 nm, or more preferably 50 to 60 nm, or 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm 110 nm, 150 nm, 200 nm, 210 nm, 250 nm, 300 nm or any range or value there between. In some embodiments, a catalyst containing a plurality of hollow zeolite particles has a bimodal particle size distribution with a first distribution having particles with an average particle size of 20 to 100 nm, 30 to 80 nm, or more preferably 50 to 60 nm, or 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm or any range or value there between and a second distribution having particles with an average particle size of 100 to 400 nm, or 150 to 350 nm, or 200 to 300 nm. A thickness of the peripheral shell can range from 5 to 30 nm, preferably 10 nm to 20 nm, or 5 nm, 6 nm, 7 nm, 8 nm, 9 nm, 10 nm, 11 nm, 12 nm, 13 nm, 14 nm, 15 nm, 16 nm, 17 nm, 18 nm, 20 nm, 21 nm, 22 nm, 23 nm, 24 nm, 25 nm, 26 nm, 27 nm, 28 nm, 29 nm, 30 nm, or any value or range there between. The particle size and thickness of the hollow space can be measured using TEM. Shell 12 includes an inner surface 13 and outer surface 18 (See, FIGS. 1C and 1D). Inner surface 15 forms the outer surface of the intra-particle hollow space 16. Inner surface 15 and outer surface 18 are made of the same zeolite material, or a combination of zeolite materials.

A plurality of the hollow zeolite particles 10 can be used to together to form a catalytic material 15. FIG. 1E depicts a plurality of hollow zeolite particles 10 in combination with an inert surface 17. Inert surface 17 can be a holder (e.g., tray, tube, etc.) or a material (e.g., binder, clays, polymeric material, etc.) that holds the hollow zeolite particles in position so that they can be used in a reaction zone. When two or more hollow zeolite particles 10 are positioned next to each other inter-particle void 19 is formed. In some instances the inert surface imparts structural integrity to the hollow zeolite particle.

### 1. Metal Nanostructure, Oxide or Alloy Thereof

Nanostructure(s) 14 can include one or more two or more active (catalytic) metals to promote the reforming of methane to carbon dioxide. The nanostructure(s) 14 can include one or more transition metals or post transition metals of the Periodic Table capable of having an oxidation state value from +2 to +7, preferably from +2 to +5, and more preferably from +2 to +3, or +2, +3, +4, +5, +6, or +7. The metals can be obtained from metal precursor compounds. Non-limiting examples of transition metal include lanthanides (Ln), titanium (Ti), zirconium (Zr), vanadium (V), niobium (Nb), chromium (Cr), molybdenum (Mo), tungsten (W), manganese (Mn), iron (Fe), rhenium (Re), ruthenium (Ru), osmium (Os), cobalt (Co), rhodium (Rh), nickel (Ni), iridium (Ir), palladium (Pd), platinum (Pt), copper (Cu), gold (Au), zinc (Zn), cadmium (Cd), mercury (Hg) or any combination thereof. Non-limiting examples of post transition metals include aluminum (Al), gallium (Ga), indium (In), tin (Sn), lead (Pb), titanium (Ti), bismuth (Bi), or any combination thereof. In a particular instance, vanadium (IV) oxide, vanadium (V) oxide, iron (II) or (III) oxide, aluminum (III) oxide, gallium (III) oxide, niobium (III) oxide, or titanium (IV) oxide, or any combination thereof can be used. For example, the metals can be obtained as a metal nitrate, a metal amine, a metal chloride, a metal coordination complex, a metal sulfate, a metal phosphate hydrate, metal complex, or any combination thereof. Examples of metal precursor compounds include vanadium chloride, vanadium nitrate, iron nitrate, iron chloride, indium nitrate, indium chloride, aluminum nitrate, aluminum chloride, gallium nitrate hydrate, gallium trichloride, or niobium chloride, titanium isopropoxide etc. These metals or metal compounds can be purchased from any chemical supplier such as Sigma-Aldrich (St. Louis, Mo., USA), Alfa-Aeaser (Ward Hill, Mass., USA), and Strem Chemicals (Newburyport, Mass., USA).

The amount of nanostructure catalyst depends, inter alia, on the use of the catalysts (e.g., alkylation of hydrocarbons). In some embodiments, the amount of catalytic metal present in the particle(s) in the hollow ranges from 0.01 to 100 parts by weight of catalyst per 100 parts by weight of catalyst, from 0.01 to 5 parts by weight of catalyst per 100 parts by weight of catalyst. If the catalyst includes more than one metal (e.g., M¹, M², and M³), M¹ and M² are each 1 to 20 weight % of the total weight of the bimetallic nanostructure or wherein M¹, M², and M³ are each 1 to 20 weight % of the total weight of the trimetallic nanostructure, based on the total weight of the catalyst. A molar amount of each bimetallic or trimetallic metal (e.g., M¹ and M² or M¹, M², and M³) in the nanostructure 14 can range from 1 to 95 molar %, or 10 to 80 molar %, 50 to 70 molar % of the total moles of the bimetallic nanostructure. The average particle size of the metal nanostructure 14 can range from 0.6 nm to 50 nm, preferably 0.6 nm to 30 nm, or more preferably 0.6 nm to 15 nm or most preferably ≤10, with the proviso that the metal nanostructure is larger than the zeolite pore size.

### 2. Zeolite Material

The zeolite shell 12 can be any porous zeolite or zeolite-like material. Zeolites belong to a broader material category known as “molecular sieves” and are often referred as such. Zeolites have uniform, molecular-sized pores, and can be separated based on their size, shape and polarity. For example, zeolites may have pore sizes ranging from about 0.3 nm to about 1 nm. The crystalline structure of zeolites can provide good mechanical properties and good thermal and chemical stability. The zeolite material can be a naturally occurring zeolite, a synthetic zeolite, a zeolite that have other materials in the zeolite framework (e.g., phosphorous), or combinations thereof. X-ray diffraction (XRD) analysis and scanning electron microscopy (SEM) may be carried out to determine the properties of zeolite materials, including their crystallinity, size and morphology. The network of such zeolites is made up of SiO₄ and/or AlO₄ tetrahedra, which are joined via shared oxygen bridges. An overview of the known structures may be found, for example, in W. M. Meier, D. H. Olson and Ch. Baerlocher, “Atlas of Zeolite Structure Types”, Elsevier, 5th edition, Amsterdam 2001. Non-limiting examples of zeolites include ABW, ACO, AEI, AEL, AEN, AET, AFG, AFI, AFN, AFO, AFR, AFS, AFT, AFX, AFY, AHT, ANA, APC, APD, AST, ATN, ATO, ATS, ATT, ATV, AWO, AWW, BEA, BIK, BOG, BPH, BRE, CAN, CAS, CFI, CGF, CGS, CHA, CHI, -CLO, CON, CZP, DAC, DDR, DFO, DFT, DOH, DON, EAB, EDI, EMT, EPI, ERI, ESV, EUO, *EWT, FAU, FER, GIS, GME, GOO, HEU, IFR, ISV, ITE, ITH, ITG, JBW, KFI, LAU, LEV, LIO, LOS, LOV, LTA, LTL, LTN, MAZ, MEI, MEL, MEP, MER, MFS, MON, MOR, MSO, MTF, MFI MTN, MTT, MTW, MWW, NAT, NES, NON, OFF, OSI, PAR, PAU, PHI, RHO, RON, RSN, RTE, RTH, RUT, SAO, SAT, SBE, SBS, SBT, SFF, SGT, SOD, STF, STI, STT, TER, THO, TON, TSC, VET, VFI, VNI, VSV, WIE, WEN, YUG and ZON structures and mixed structures of two or more of the abovementioned structures. In some embodiments, the zeolite includes phosphorous to form a AIPOx structure with the appropriate porosity. Non-limiting examples of AIPOx zeolites include AABW, AACO, AAEI, AAEL, AAEN, AAET, AAFG, AAFI, AAFN, AAFO, AAFR, AAFS, AAFT, AAFX, AAFY, AAHT, AANA, AAPC, AAPD, AAST, AATN, AATO, AATS, AATT, AATV, AAWO, AAWW, ABEA, ABIK, ABOG, ABPH, ABRE, ACAN, ACAS, ACFI, ACGF, ACGS, ACHA, ACHI, A-CLO, ACON, ACZP, ADAC, ADDR, ADFO, ADFT, ADOH, ADON, AEAB, AEDI, AEMT, AEPI, AERI, AESV, AEUO, A*EWT, AFAU, AFER, AGIS, AGME, AGOO, AHEU, AIFR, AISV, AITE, AITH, AITG, AJBW, AKFI, ALAU, ALEV, ALIO, ALOS, ALOV, ALTA, ALTL, ALTN, AMAZ, AMEI, AMEL, AMEP, AMER, AMFI, AMFS, AMON, AMOR, AMSO, AMTF, AMTN, AMTT, AMTW, AMWW, ANAT, ANES, ANON, AOFF, AOSI, APAR, APAU, APHI, ARHO, ARON, ARSN, ARTE, ARTH, ARUT, ASAO, ASAT, ASBE, ASBS, ASBT, ASFF, ASGT, ASOD, ASTF, ASTI, ASTT, ATER, ATHO, ATON, ATSC, AVET, AVFI, AVNI, AVSV, AWIE, AWEN, AYUG and AZON structures and mixed structures of two or more of the abovementioned structures. In particular embodiments, the zeolite is a porous zeolite in pure silica (Si/Al=∞) form or with a small amount of Al, for example, *BEA, MFI, silicalite-1, type Y or combinations thereof zeolites. The zeolite can have a Si/Al of 500, 550, 600, 650, 700, 750, 800, 850, 900, 950, 100, up to ∞, or any value or range there between. In some instances the *BEA can include fluoride ions (e.g., *BEA synthesized using fluoride media), where F ions are substituted with aluminum ions in the crystal lattice. The zeolite of the present invention is a pure porous zeolite having none or substantially no acidic sites on the surface of the zeolite. In a particular aspect, the zeolite is not ZSM-5 or H-ZSM-5. The zeolite can be organophilic. Zeolites may be obtained from a commercial manufacturer such as Zeolyst (Valley Forge, Pa., U.S.A.).

### B. Preparation Encapsulated Metal Nanostructure/Hollow Zeolite Material

Catalytic materials exist in various forms and their preparation can involve multiple steps. The catalysts can be prepared by processes known to those having ordinary skill in the art, for example, the catalyst can be prepared by any one of the methods comprising liquid-liquid blending, solid-solid blending, or liquid-solid blending (e.g., any of precipitation, co-precipitation, impregnation, complexation, gelation, crystallization, microemulsion, sol-gel, solvothermal, dissolution-recrystallization, hydrothermal, sonochemical, or combinations thereof).

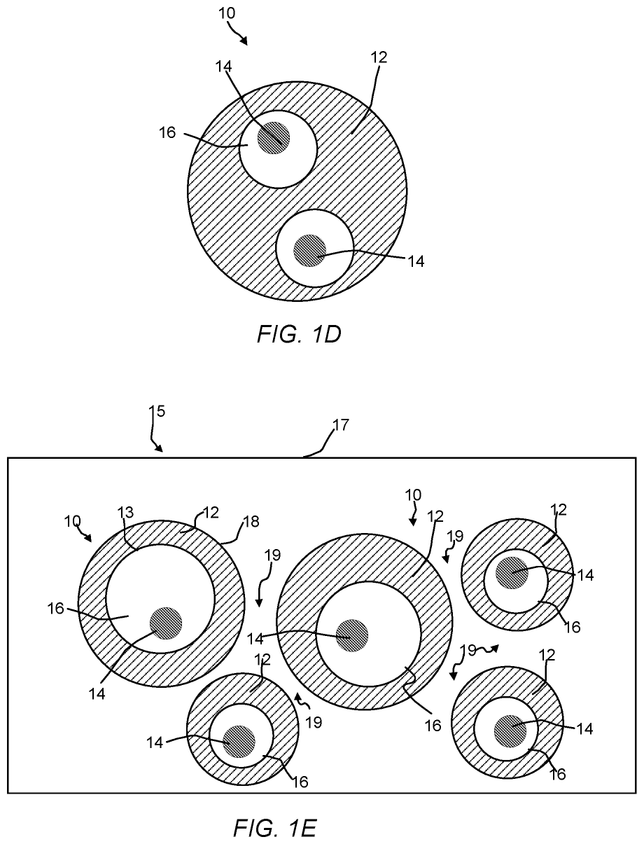

FIGS. 2A and 2B are a schematic of methods to make an encapsulated metal nanoparticle/hollow shell zeolite material or multiple nanostructures in a hollow shell zeolite. In method 20, step 1, the zeolite material 22 can be either obtained through a commercial source or prepared using the methods described in the Examples section. An aqueous solution of the metal precursor material (e.g., an iron precursor), or a combination of metal precursors (e.g., to make multiple nanostructures in the hollow zeolite as shown in FIG. 2B) can be contacted with the zeolite material to allow impregnation of the zeolite material with the precursor materials 24. The amount of solution of metal precursor material is the same or substantially the same as the pore volume of the zeolite material. The impregnated zeolite material can be dried to obtain a metal (e.g., mono-, bi- or tri-metallic) impregnated zeolite material 26. Drying conditions can include heating the impregnated zeolite material 26 from 30° C. to 100° C., preferably 40° C. to 60° C., for 4 to 24 hours. In step 2, the impregnated zeolite material 26 can be contacted (suspended) with an aqueous solution of a templating agent (e.g., a quaternary ammonium hydroxide compound) and the resulting suspension is subjected to a dissolution-recrystallization process to produce the encapsulated nanoparticle/zeolite composite material 28 having metal nanostructure 24 (FIG. 2A) or nanostructures (FIG. 2B) positioned in hollow 30. In some embodiments, the zeolite is subjected to a vacuum prior to impregnation (e.g., 100 to 300° C. for 6 h under 10⁻⁶ bar) to facilitate metal diffusion through the pores and/or to remove any Brønsted acid sites. The dissolution-recrystallization process under hydrothermal conditions can include techniques of heating aqueous solutions of the aqueous templated zeolite suspension at high vapor pressures. In a particular embodiment, the suspension is heated to 100° C. to 250° C., preferably 150° C. to 200° C., for 12 to 36 hours, preferably 18 to 30 hours under autogenous pressure. Dissolution-recrystallization can performed in a pressure vessel, such as an autoclave, by a temperature-difference method, temperature-reduction method, or a metastable-phase technique. Without wishing to be bound by theory, it is believed that during the dissolution-recrystallization process, the hollow is formed in the zeolite framework through dissolution of some of the silicon core by the templating agent. The removed silica species can recrystallize on the outer surface upon cooling. During the hydrothermal process, the metal precursors can form a metal nanostructure in the intra-particle hollow space. Since the metal particles are too large to migrate through the microporous zeolite walls, they remain in the hollow space. In some instances, small nanostructures come together and form a larger nanostructure or a single nanostructure in the hollow space. In step 3, the resulting metal-zeolite composite material 28 can be heated in the presence of air (e.g., calcined) to remove the template and any organic residues to form an encapsulated metal nanostructure/ hollow zeolite material 10. The zeolite material has metal nanostructure(s) 14 in an intra-particle hollow space 14 in the zeolite shell 12. Calcination conditions can include a temperature of 350° C. to 550° C., preferably 400° C. to 500° C. and a time of 3 to 10 hours, preferably 4 to 8 hours.

### C. System and Method for Production of Ethylbenzene from Ethylene and Benzene

Also disclosed are systems and methods of producing alkyl aromatics from aromatic compounds and olefinic compounds (e.g., ethylbenzene from benzene and ethylene or cumene from benzene and propylene) under Lewis acid alkylation conditions. Alkylation conditions can include contacting the catalyst materials 10 discussed above and/or throughout this specification having an active Lewis acid site (e.g., iron (II) or (III) oxide) with the olefin (e.g., ethylene, propylene, etc.) and the aromatic compound (e.g., benzene) under sufficient conditions to produce an alkyl aromatic compound (e.g., ethylbenzene, cumene, etc.). Such conditions sufficient to produce the gaseous mixture can include a temperature range of 150° C. to 400° C. from 200° C. to 350° C. or from 250° C. to 300° C. or 150° C., 175° C., 200° C., 225° C., 250° C., 275° C., 300° C., 325° C., 350° C., 375° C., 400° C., or any value there between and a pressure range of about 5 bara (0.5 MPa) to 70 bara (7 MPa), and/or a gas hourly space velocity (GHSV) ranging from 1,000 to 100,000 h⁻¹. In certain aspects, the carbon formation or coking is reduced or does not occur on the catalyst material 10, leaching is reduced or does not occur, and/or sintering is reduced or does not on the catalyst material 10. Furthermore, mono-substituted alkyl aromatic products are obtained in greater than 90 wt. %, 95 wt. % or 99.9 wt. % based on the weight of the total product stream. In a particular instance benzene selectivity can be 90 wt. %, 99.9 wt. % or 100 wt. %. This is in contrast to conventional methods, which produces multi-substituted by-products (e.g., di-, tri-, and tetra-substituted aromatic compounds).

In instances when the produced catalytic material is used in alkylation reactions, the olefin can be obtained from various sources. In one non-limiting instance, ethylene or propylene can be obtained from steam cracking of hydrocarbons. The aromatic hydrocarbon material used in the reaction can be benzene. Benzene can be obtained from catalytic reforming of hydrocarbons, toluene hydrodealkylation, toluene disproportionation, and steam cracking. The resulting ethylbenzene or cumene can then be used in additional downstream reaction schemes to create additional products. Such examples include chemical products such as styrene or polystyrene productions. Notably, the product mixture includes none or substantially no by-products (e.g., diethylbenzene, triethylbenzene, diphenyl ethane, or other polyalkylated benzenes).

The method can further include isolating and/or storing the produced mixture. The method can also include separating unreacted ethylene or propylene from the produced liquid mixture and/or heavier reaction products from the ethylbenzene or cumene.

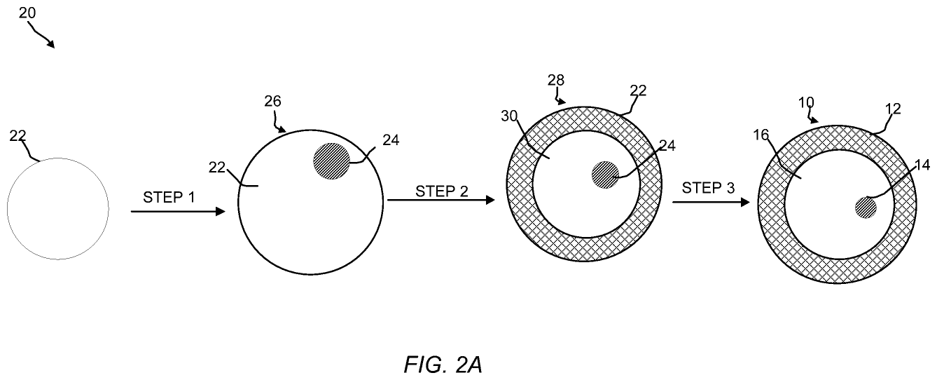

Systems for producing an alkyl aromatic compound (e.g., ethylbenzene, cumene, etc.) from an aromatic hydrocarbon (e.g., benzene) and an olefinic compound (e.g., ethylene, propylene, etc.) are also described. FIG. 3 depicts a schematic for a system to produce an alkyl aromatic compound. The system 30 can include an inlet 32 for an aromatic compound reactant feed, an inlet 34 for an olefinic reactant feed, a reaction zone 36 (e.g., a continuous flow reactor selected from a fixed-bed reactor, a fluidized reactor, or a moving bed reactor) that is configured to be in fluid communication with the inlets 32 and 34; and an outlet 38 configured to be in fluid communication with the reaction zone 34 and configured to remove a product stream from the reaction zone. The reactant zone 34 can include catalyst of the present invention. The aromatic compound can enter the reaction zone 36 via the aromatic compound inlet 32. After a sufficient amount of aromatic compound and catalyst have been placed in the reaction zone 36, a gaseous stream that includes the olefinic compound can enter the reaction zone through the olefinic compound feed inlet 34. The olefinic compound can be used to maintain a pressure in the reaction zone 36 from 5 bar to 50 bar. In some embodiments, the olefinic compound feed stream includes inert gas (e.g., nitrogen or argon). After a sufficient amount of time, the liquid product stream can be removed from the reaction zone 36 via product outlet 38. The product stream can be sent to other processing units, stored, and/or transported.

### EXAMPLES

The present invention will be described in greater detail by way of specific examples. The following examples are offered for illustrative purposes only, and are not intended to limit the invention in any manner. Those of skill in the art will readily recognize a variety of noncritical parameters, which can be changed or modified to yield essentially the same results.

### Example 1

### Synthesis of Fe(III)₂O₃/Hollow-Silicalite-1 Catalyst Material

Silicalite-1 is obtained by mixing tetraethylorthosilicate (TEOS, 98% purity, Sigma-Aldrich®, USA) and tetrapropylammonium hydroxide (TPA(OH), 1.0 M, in H₂O, Sigma-Aldrich®, USA) with water. The gel composition is SiO₂: 0.4 TPA(OH): 35 H₂O. Then, the mixture is transferred into a Teflon-lined autoclave and heated at 170° C. under static condition for 3 days. The solid was recovery by centrifugation and washed with water, which was repeated 3 times. The resulting solid was dried overnight at 110° C. and then calcined at 525° C. in air for 12 h. Subsequently, the zeolite was treated under vacuum (0.1 to 1 mbar) at 300° C. for 10 hours. Then, dry impregnation of iron nitrate on the zeolite surface was carried out (3 wt. % of Fe, C_{Fe(NO3)3 solution}=0.5 mol. L⁻¹) in water. After impregnation, the impregnated zeolite was dried and then calcined (2 h under air, 400° C., 1° C./min) in order to obtain a metal oxide which was insoluble. While the metal oxide particles were well dispersed, the zeolite was treated with the corresponding template in the hydroxide form, tetrapropylammonium hydroxide (TPA(OH), (Sigma-Aldrich®, USA) for MFI structure. The mixture was transferred into a Teflon-lined autoclave and heated at 170° C. under static conditions for 24 h. The material was recovered by centrifugation and washed 3 times with water to remove the excess of template. After drying the material at 100° C. under air for 10 hours, the zeolite was calcined at 500° C., (1° C./min) under air for 6 hour obtain the zeolite of the present invention with clean pores.

### Example 2

### Characterization of Fe₂O₃ in Hollow Silicalite-1

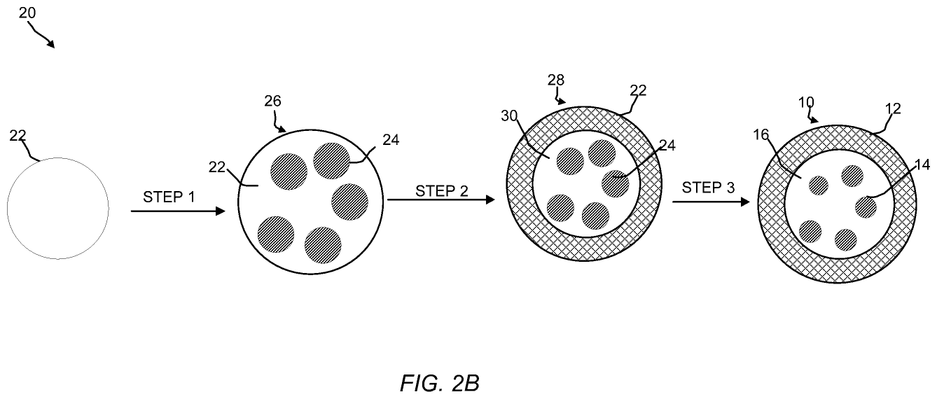

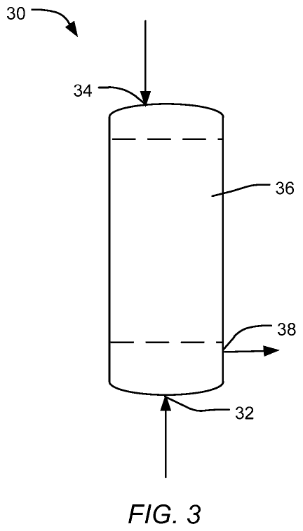

Transmission Electron Microscopy (TEM) analysis was performed on the inventive sample from Example 1 using a Titan G2 80-300 kV transmission electron microscope operating at 300 kV (FEI™, USA) equipped with a 4 k×4 k CCD camera, a GIF Tridiem filter (Gatan, Inc., USA), and an Energy dispersive X-ray (EDAX) detector. FIGS. 4A and 4B are TEM images of the catalyst of Example 1 at a scale of 200 nm and 20 nm. From the TEM, it was determined that regular hexagonal shaped nanoparticles of hollow zeolite with an average dimension of 50 to 60 nm were formed. The wall thickness of the hollow silicalite-1 zeolite was about 15 nm whereas the cavity was about 30 nm. Nanoparticles of iron were not observable within the microscope resolution, but were detected by EDAX (See, FIG. 5). It was determined that by using this process of making the metal nanostructure/hollow zeolite, a high homogeneity of hollow zeolite was obtained in terms of size and shape. From these characterizations, we can conclude that Fe-silicalite-1 was obtained.

### Example 3

### Production of Ethylbenzene from Ethylene and Benzene

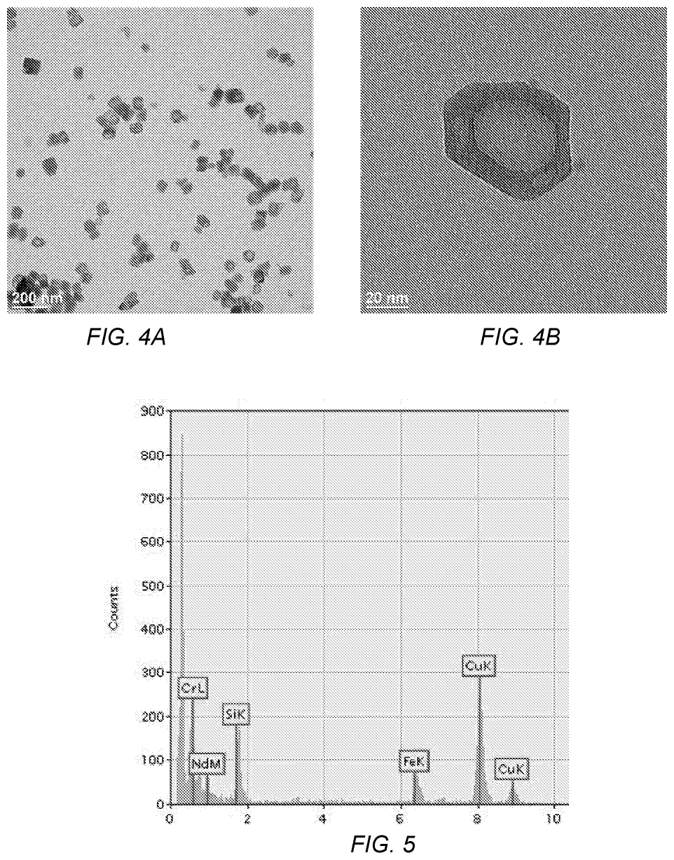

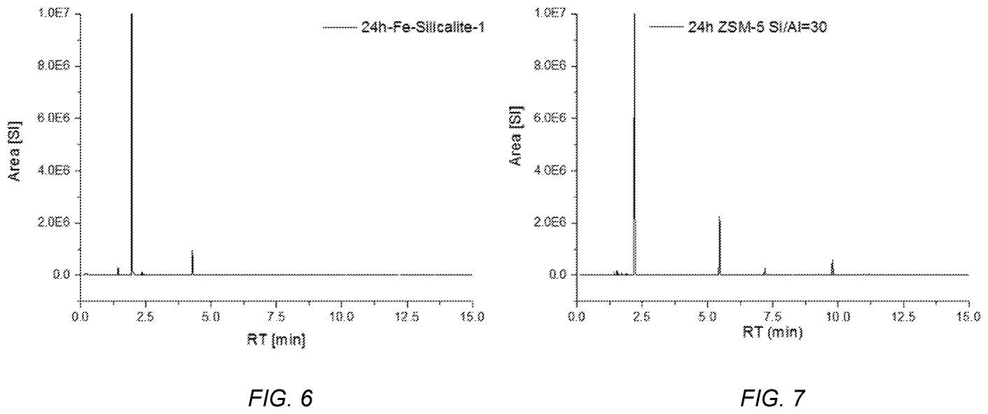

The catalyst (300 g) from Example 1 or a comparative catalyst (H-ZSM-5, Si/Al=30) and benzene (10 mL) were introduced into a 100 mL PARR autoclave reactor. Then, the pressure was increased to 10 bar with pure ethylene. The reactor was stirred and the temperature was increased to 250° C. After 24 h, the reaction was cooled and the liquid phase was analyzed by using an Agilent Technologies (USA) GC-MS (Agilent 7890b with a FID detector and HP 5MS UI columns, 0.25 micrometer and a Agilent 5977A Mass spectrometer). The benzene conversion for the comparative catalyst 15% and the conversion for the Fe-silicate-1 catalyst of the present invention was 9%. FIG. 6 shows a high selectivity in ethylbenzene (Selec_{EB}) of 90% for the Fe-Silicalite-1 catalyst. In FIG. 6, the peak at 2.2 min. was benzene and the peak at 4.3 min. was ethylbenzene. FIG. 7 shows the comparative catalyst with the formation with ethylbenzene and the by-product methylpropylbenzene. In FIG. 7, the peak at 2.2 min. was benzene, the peak 5.4 min. was ethylbenzene and the peak at the 9.8 min was 1-methylpropylbenzene. Under these experimental conditions, a comparative ZSM5-Si/Al=30 without a metal was less selective than the Fe-Silicalite-1 with almost the similar benzene conversion, 15% and 9% respectively.

1. A supported catalyst comprising:
(a) a metal nanostructure, an oxide, or an alloy thereof, having a Lewis acid active site capable of catalyzing formation of an alkyl aromatic compound from an aromatic hydrocarbon and an olefin; and
(b) an inert hollow zeolite support having a peripheral shell with an exterior surface and an interior surface that defines and encloses a hollow space within the interior of the shell, wherein the metal nanostructure, or an oxide or an alloy thereof, is comprised in the hollow space.

2. The supported catalyst of claim 1, wherein the alkyl aromatic compound is ethylbenzene, the aromatic hydrocarbon is benzene, and the olefin is ethylene or wherein the alkyl aromatic compound is cumene, the aromatic hydrocarbon is benzene, and the olefin is propylene.

3. The supported catalyst of claim 1, wherein the hollow zeolite support is a *BEA, MFI, silicalite-1, or any combination thereof.

4. The supported catalyst of claim 3, wherein the hollow zeolite support Si/Al ratio of 500 to infinity (∞).

5. The supported catalyst of claim 3, wherein the hollow zeolite support is a pure *BEA zeolite support, wherein the *BEA comprises fluoride ions.

6. The supported catalyst of claim 3, wherein the hollow zeolite support is a pure MFI zeolite support, preferably pure silicalite-1.

7. The supported catalyst of claim 1, wherein the metal of the metal nanostructure, oxide or alloy thereof is a transition metal, a post transition metal, or both, having an oxidation state value from +2 to +7, preferably from +2 to +5, and more preferably from +2 to +3.

8. The supported catalyst of claim 7, wherein the metal nanostructure is vanadium (IV) oxide, vanadium (V) oxide, iron (II) or (III) oxide, niobium (III) oxide, aluminum (III) oxide, gallium (III) oxide, titanium (IV) oxide, or any combination thereof.

9. The supported catalyst of claim 1, wherein the crystal structure of the metal nanostructure oxide phases or phases is a mono oxide, a composite oxide, or a solid solution of mixed oxide.

10. The supported catalyst of claim 1, wherein the metal nanostructure, or oxide or alloy thereof is 0.5 to 20 wt. %, preferably 1 to 10 wt. %, of the supported catalyst and the hollow zeolite support is 80 to 99.5 wt. % of the supported catalyst.

11. The supported catalyst of claim 1, wherein the hollow space comprises a single metal nanostructure or oxide thereof or alloy thereof.

12. The supported catalyst of claim 1, wherein the hollow space comprises a plurality of the metal nanostructures or oxides thereof or alloy thereof.

13. The supported catalyst of claim 1, wherein the metal nanostructure, or oxide or alloy thereof, is deposited on the interior surface of the peripheral shell.

14. The supported catalyst of claim 1, wherein the size of the hollow space and the metal nanostructure, or oxide or alloy thereof, are both larger than the average pore size of the pores in the hollow zeolite support.

15. The supported catalyst of claim 14, wherein:
the hollow zeolite is a single particle having a particle size of 20 nm to 300 nm, preferably 20 nm to 100 nm;
the thickness of the peripheral shell is 5 nm to 30 nm;
the volume of the hollow space is 5% to 90% of the initial particle volume; and/or
the particle size of the metal nanostructure or oxide or alloy thereof is larger than the zeolite average pore size and is 0.6 to 50 nm.

16. The supported catalyst of claim 1, wherein the metal nanostructure is not an iron-potassium (FeK) containing metal nanostructure and/or the hollow zeolite support is not a ZSM-5 support.

17. A method for producing alkyl aromatic compound comprising contacting the supported catalyst of claim 1 with an aromatic hydrocarbon and olefin in a reaction zone under reaction conditions sufficient to produce the alkyl aromatic compound.

18. The method of claim 17, wherein the alkyl aromatic compound is ethylbenzene, the aromatic hydrocarbon is benzene, and the olefin is ethylene or wherein the alkyl aromatic compound is cumene, the aromatic hydrocarbon is benzene, and the olefin is propylene.

19. A method of making the supported catalyst of claim 1, the method comprising:
(a) obtaining a zeolite support;
(b) obtaining a first suspension by suspending the zeolite support in an aqueous solution having a metal nanostructure precursor material for a sufficient period of time to impregnate the support with the precursor material and drying the first suspension to obtain an impregnated support;
(c) obtaining a second suspension by suspending the impregnated support from step (b) in an aqueous solution comprising a templating agent and thermally treating the suspension to obtain a templated support; and
(d) calcining the templated support to obtain the supported catalyst of claim 1.

20. The method of claim 19, wherein:
the metal nanostructure precursor material is a metal nitrate, a metal amine, a metal halogen, a metal coordination complex, a metal sulfate, a metal phosphate hydrate, or combination thereof;
drying the first suspension to obtain the impregnated support in step (b) comprises subjecting the first suspension to a temperature of 30° C. to 100° C., for 2 to 24 hours;
thermally treating the second suspension to obtain the templated support in step (c) comprises subjecting the second suspension to a temperature of 100° C. to 250° C. C, for 12 to 96 hours; and/or
calcining step (d) comprises subjecting the templated support to a temperature of 400° C. to 600° C. for 3 to 10 hours.
